(19) United States
(12) Patent Application Publication
(10) Pub. No.: US 2009/0209993 A1
(43) Pub. Date: Aug. 20, 2009

(54) THERAPY DEVICE

(76) Inventor: Chin-Chih HSIEH, Yunghe City (TW)

Correspondence Address: HDLS Patent & Trademark Services, P.O. BOX 220746, CHANTILLY, VA, 20153-0746, US

(21) Appl. No.: 12/033,259

(22) Filed: Feb. 19, 2008

(51) Int. Cl. A61B 17/34; A61N 1/00

(52) U.S. Cl. 606/189; 607/46

(57) ABSTRACT

A therapy device includes an electric acupuncture band and an electric acupuncture element. The electric acupuncture band is provided with an electric acupuncture portion and a bonding portion connected to the electric acupuncture portion. The electric acupuncture portion is formed by means of joining an accommodating layer and an electric acupuncture layer. The electric acupuncture element comprises an electric acupuncture piece provided between the accommodating layer and the electric acupuncture layer of the electric acupuncture band. The surface of the electric acupuncture piece is exposed to the outside of the electric acupuncture layer.

### BACKGROUND OF THE INVENTION

1. Field of the Invention

The present invention relates to a therapy device, and in particular to a low-frequency therapy device that can be applied to extremities of a human body.

2. Description of Prior Art

Recently, with the improvement of the living standard, people pay more attention to take care of their health. After working for a long time and getting so tired, people like to relax their body by means of acupuncture or massage.

The acupuncture and massage can stimulate the acupuncture points of a human body directly, thereby adjusting the body function, increasing immunity, and even curing diseases. However, the acupuncture is so specialized that ordinary people cannot do it at home. Although the massage can be performed at many times and places, the effect of massage may insufficient because of bad control of applied forces or lack of perseverance and willpower. Therefore, a low-frequency therapy device is available in the market, by which a weak current is generated to stimulate acupuncture points so as to relax muscles and adjust personal body function.

FIG. 1 shows the structure of a conventional low-frequency therapy device. The therapy device 1 is constituted of an electric acupuncture machine 11, an electric acupuncture element 13 and a conductive patch 15. The electric acupuncture machine 11 includes a displaying section 111 and an operating section 113 for operating the information displayed in the displaying section 111. One end of the electric acupuncture element 13 is electrically connected to the electric acupuncture machine 11, and the other end thereof is provided with an electric acupuncture piece 131. The electric acupuncture piece 131 is disposed on the conductive patch 15. In use, the conductive patch 15 is attached to the skin of a human body. Then, the electric acupuncture machine 11 generates a moderate current to flow through the electric acupuncture element 13 and the conductive patch 15, thereby stimulating, massaging and treating the acupuncture points of a human body.

However, when such kind of low-frequency therapy device 1 is in operation, a user has to attach the conductive patch 15 to the skin of a human body, and hold the electric acupuncture machine 11 or put it in a pocket of the clothes. Therefore, it is inconvenient for a moving user to operate or transport. On the other hand, since the electric acupuncture machine 11 and the electric acupuncture element 13 are stored separately, the risk of losing the electric acupuncture machine 11 or the electric acupuncture element 13 is increased.

Therefore, it is an important issue to propose an improved therapy device to overcome the drawbacks of prior art.

### SUMMARY OF THE INVENTION

The present invention is to provide a therapy device, in which a machine, an electric acupuncture element, and an electric acupuncture band are integrated into a unit for easy use, transportation and storage.

The present invention provides a therapy device which is constituted of an electric acupuncture band and an electric acupuncture element. The electric acupuncture band is provided with an electric acupuncture portion and a bonding portion connected to the electric acupuncture portion. The electric acupuncture portion is formed by means of joining an accommodating layer and an electric acupuncture layer. The electric acupuncture element comprises an electric acupuncture piece provided between the accommodating layer and the electric acupuncture layer of the electric acupuncture band. The surface of the electric acupuncture piece is exposed to the outside of the electric acupuncture layer.

Also, the present invention provides a therapy device which is constituted of an electric acupuncture band, a machine and an electric acupuncture element. The electric acupuncture band is provided with an electric acupuncture portion and a bonding portion connected to the electric acupuncture portion. The electric acupuncture portion is formed by means of joining an accommodating layer and an electric acupuncture layer. The machine can be mounted on the accommodating layer. The machine has a displaying section and an operating section for operating the information displayed in the displaying section. One end of the electric acupuncture element is electrically connected to the machine, and the other end thereof is provided with an electric acupuncture piece. The electric acupuncture piece is provided between the accommodating layer and the electric acupuncture layer of the electric acupuncture band, and is exposed to the outside of the electric acupuncture layer.

With the integration of the machine, the electric acupuncture element and the electric acupuncture band into one unit, the problems occurred in prior art, such as the inconvenience in operation and storage, and the risk of losing the machine or electric acupuncture element, can be solved. Therefore, the present invention brings about convenience in use, transportation and storage, and thus really has industrial applicability.

### BRIEF DESCRIPTION OF THE DRAWINGS

FIG. 1 is a view showing the structure of a conventional low-frequency therapy device;

FIG. 2 is a perspective view of the present invention;

FIG. 3 is a partially exploded view of the present invention;

FIG. 4 is a partially assembled view of the present invention;

FIG. 5 is a schematic view showing the operating state of the present invention;

FIG. 6 is a schematic view showing the back surface of the electric acupuncture band and the conductive patch of the present invention; and

FIG. 7 is a schematic view showing another embodiment of the present invention.

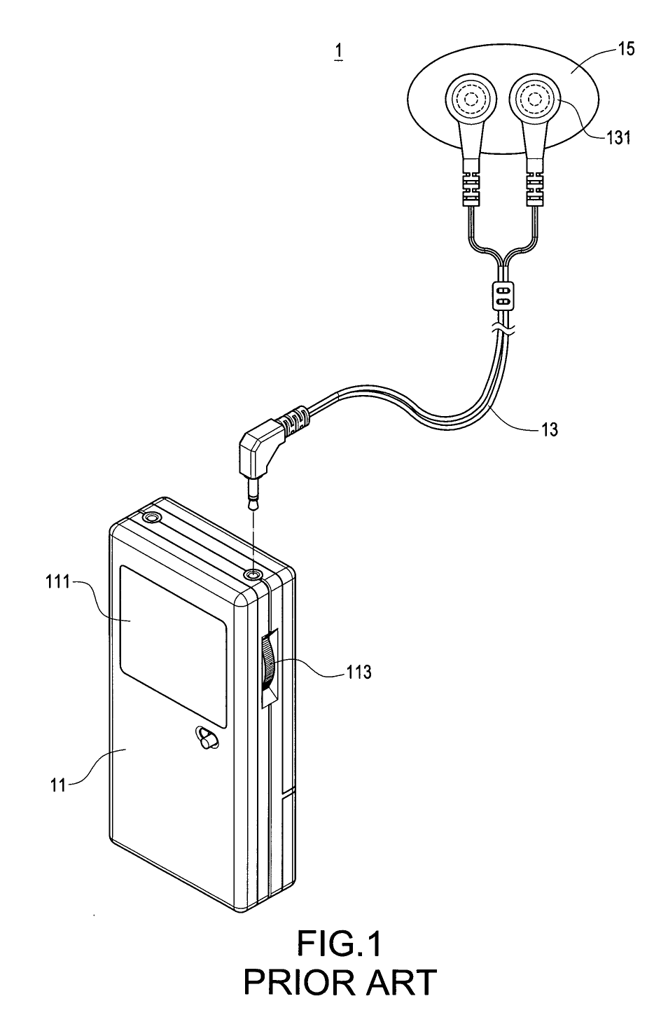

### DETAILED DESCRIPTION OF THE INVENTION

The characteristics and technical contents of the present invention will be explained with reference to the preferred embodiments thereof and the accompanying drawings. However, the embodiments and drawings are illustrative only, but not used to limit the scope of the present invention.

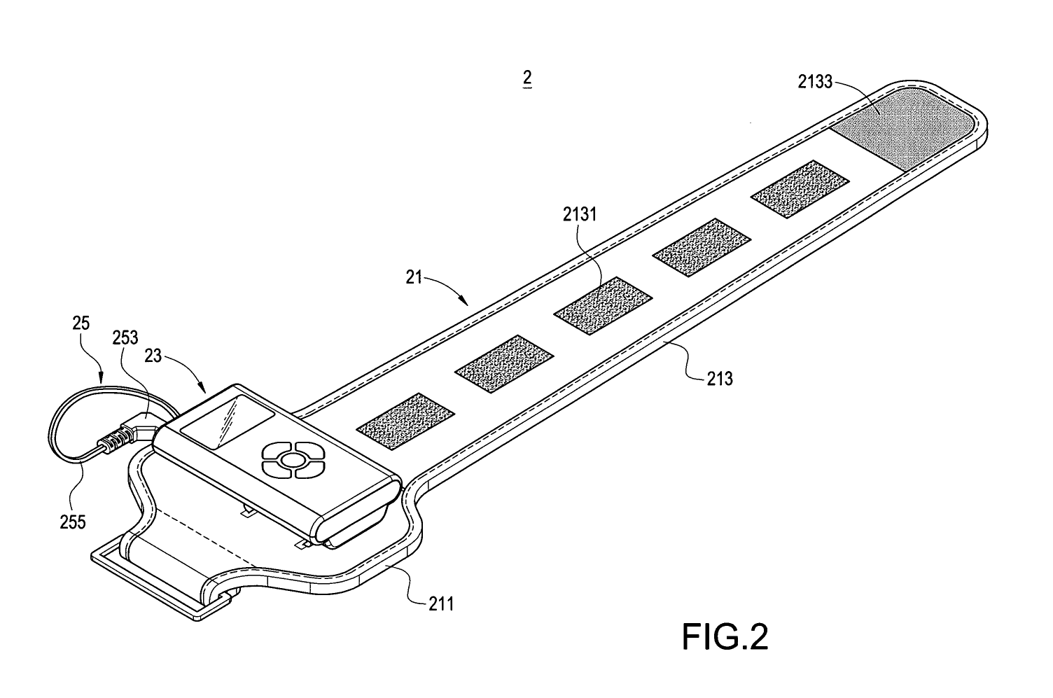

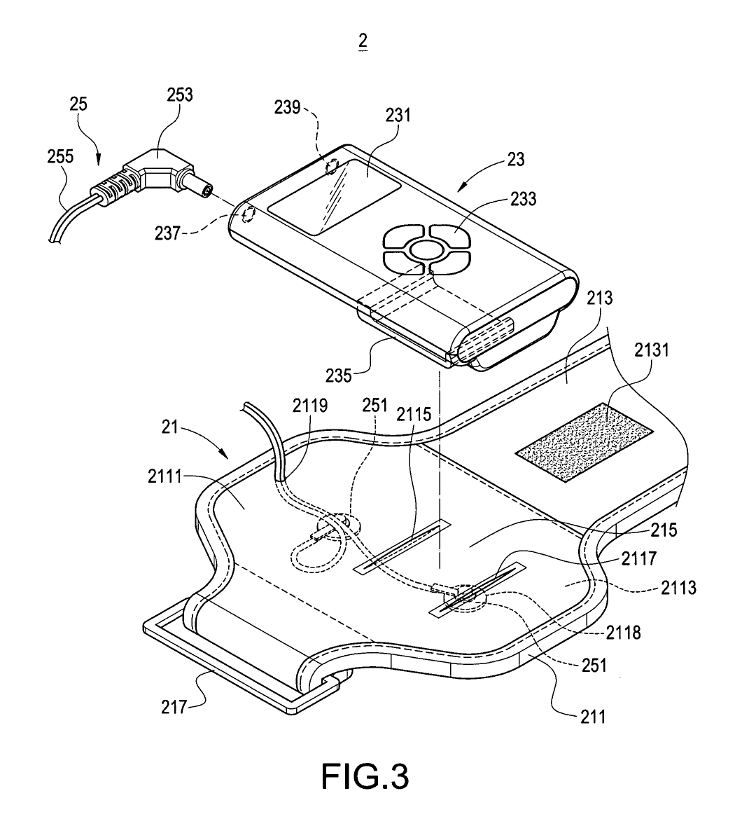

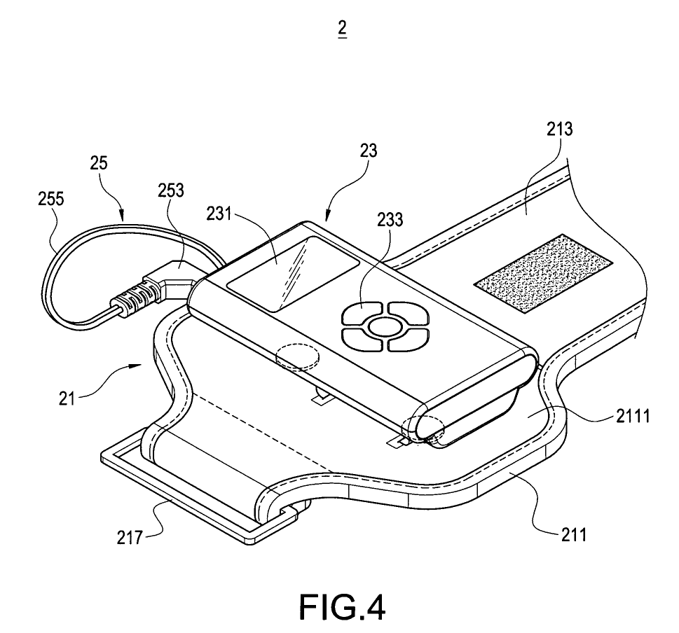

Please refer to FIGS. 2 to 4. The therapy device of the present invention is constituted of an electric acupuncture band 21, a machine 23 and an electric acupuncture element 25.

The electric acupuncture band 21 is provided with an electric acupuncture portion 211 and a bonding portion 213 connected to the electric acupuncture portion 211. The electric acupuncture portion 211 is formed by means of joining an accommodating layer 2111 and an electric acupuncture layer 2113. The bonding portion 213 is provided with a plurality of fastening surfaces 2131 in a lengthwise direction. A hairy surface 2133 is provided on one end of the bonding portion 213 far from the electric acupuncture portion 211. The hairy surface 2133 can be adhered on the fastening surface 2131. The bonding portion 213 can be an elastically stretchable band. The accommodating layer 2111 is provided with a first through hole 2115, a second through hole and a third through hole 2119. A clamping portion 215 is formed between the first through hole 2115 and the second through hole 2117. The third through hole 2119 allows the electric acupuncture element 25 to penetrate the surface of the electric acupuncture portion 211.

The machine 23 can be mounted on the accommodating layer 2111. The machine 23 has a displaying section 231 and an operating section 233 for operating the information displayed in the displaying section 231. The machine 23 is also provided with a fastener 235 for penetrating the first through hole 2115 and the second through hole 2117. The fastener 235 is clamped on the clamping portion 215. The fastener 235 can be a hook. Further, the machine 23 is also provided with a power output port 237 and an audio signal output port 239. The power output port 236 provides necessary power for the electric acupuncture piece 251, and the audio signal output port 239 is used to output the audio signals generated by the machine 23. The machine 23 can be a machine of a low-frequency therapy device, music player or the combination thereof.

The electric acupuncture element 25 comprises an electric acupuncture piece 251, a plug 253 and a lead 255 electrically connected between the electric acupuncture piece 251 and the plug 253. The plug 253 is inserted into the power output port 237 of the machine 23. The electric acupuncture piece 251 is disposed between the accommodating layer 2111 and the electric acupuncture layer 2113 of the electric acupuncture band 21. The surface of the electric acupuncture piece 251 is exposed to the outside of the electric acupuncture layer 2113. The electric acupuncture layer 2113 is provided with a plurality of holes 2118 for allowing the electric acupuncture piece 251 to be exposed to the outside.

In the present embodiment, a ring 217 is provided on one side of the electric acupuncture portion 211 so as to allow the penetration of the bonding portion 213 to secure the electric acupuncture band 21 to a region of the human body. Optionally, if there is no ring 217, the electric acupuncture band 21 can be also secured to a region of the human body in such a manner that the bonding portion 213 wraps the desired region of the human body and then wraps to cover the machine 23. Finally, the hairy surface 2113 is attached to the fastening surface 2131 at one side of the machine 23, thus completing the fixation of the therapy device.

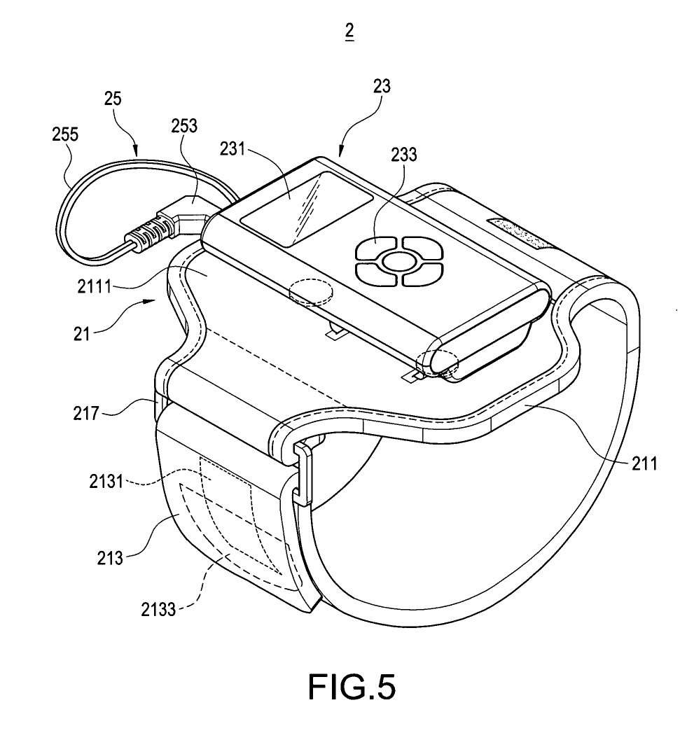

Please refer to FIG. 5. In use, the machine 23, the electric acupuncture element 23 and the electric acupuncture band 21 can be integrated into one unit. The user can wrap the electric acupuncture band 21 around the desired region of the human body, and then penetrates one end of the bonding portion 213 through the ring 217 until the length and elasticity of the electric acupuncture band 21 is adjusted to a moderate extent. Finally, the user utilizes the hairy surface 2133 to attach to the fastening surface 2131, thereby completing the fixation of the present invention. Further, for a moving user, the arrangement of the present invention makes it convenient to use and transport. In addition, since the machine 23 and the electric acupuncture element 25 can be stored together with the electric acupuncture band 21, the storage of the whole therapy device of the present invention is so convenient that the risk of losing each accessory thereof can be eliminated.

However, the previous embodiment in which the electric acupuncture band 21 wraps around the desired region of the human body is more suitable to secure the electric acupuncture band 21 to four extremities of the human body.

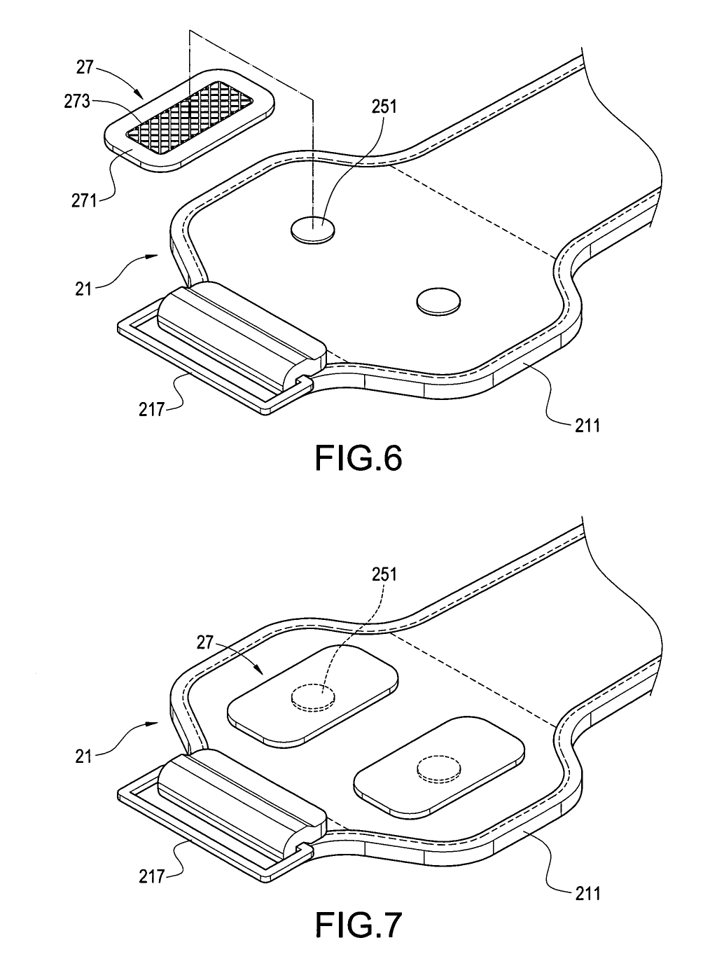

In the present embodiment, a conductive patch 27 is further provided. As shown in FIGS. 6 and 7, the conductive patch 27 includes an adhesive layer 271, a conductive layer 273, a conductive glue (not shown), and two isolation papers (not shown). Both surfaces of the adhesive layer 271 are adhesive. The conductive layer 273 is formed on one surface of the adhesive layer 271. The conductive glue is uniformly applied to the surfaces of the adhesive layer 271 and the conductive layer 273. The isolation papers are adhered on each surface of the adhesive layer 271. Therefore, if the conductive patch 27 is to be adhered on the electric acupuncture piece 251, the respective isolation papers on the conductive patch 27 have to be peeled off first. The surface of the conductive patch 27 with the conductive layer 273 thereon is adhered to the electric acupuncture piece 251, thereby achieving the electrical connection between the conductive layer 273 and the electric acupuncture piece 251. In this way, the conductive patch 27 can be attached to the skin of the human body so as to stimulate, massage and treat the acupuncture points of the human body.

According to the above, the present invention really brings about convenience in use, transportation and storage, and solves the drawbacks of prior art. Further, the present invention has novelty and inventive steps, and thus conforms to the requirements for a utility model patent.

Although the present invention has been described with reference to the foregoing preferred embodiments, it will be understood that the invention is not limited to the details thereof Various equivalent variations and modifications can still occur to those skilled in this art in view of the teachings of the present invention. Thus, all such variations and equivalent modifications are also embraced within the scope of the invention as defined in the appended claims.

What is claimed is:

1. A therapy device, comprising:
an electric acupuncture band provided with an electric acupuncture portion and a bonding portion connected to the electric acupuncture portion, the electric acupuncture portion being formed by means of joining an accommodating layer and an electric acupuncture layer; and
an electric acupuncture element comprising an electric acupuncture piece, the electric acupuncture piece being provided between the accommodating layer and the electric acupuncture layer of the electric acupuncture band, the surface of the electric acupuncture piece being exposed to the outside of the electric acupuncture layer.

2. The therapy device according to claim 1, further comprising a machine mounted on the accommodating layer and electrically connected to the electric acupuncture piece, and the machine having a displaying section and an operating section for operating the information displayed in the displaying section.

3. The therapy device according to claim 1, further comprising a ring provided on one side of the electric acupuncture portion for allowing the penetration of the bonding portion.

4. The therapy device according to claim 1, wherein the bonding portion is provided with a plurality of fastening surfaces in a lengthwise direction, a hairy surface is provided on one end of the bonding portion far from the electric acupuncture portion, and the hairy surface is detachably adhered to fastening surface.

5. The therapy device according to claim 1, wherein the bonding portion is an elastically stretchable band.

6. The therapy device according to claim 2, wherein the accommodating layer is provided with a first through and a second through hole, the machine is provided with a fastener penetrating the first through hole and the second through hole.

7. The therapy device according to claim 6, wherein the fastener is a hook.

8. The therapy device according to claim 1, wherein the accommodating layer is provided with a third through hole for allowing the electric acupuncture element to penetrate through the surface of the electric acupuncture portion.

9. The therapy device according to claim 1, wherein the electric acupuncture layer is provided with a plurality of holes for allowing the electric acupuncture piece to be exposed to the outside.

10. The therapy device according to claim 2, wherein the machine is provided with a power output port for supplying necessary electricity to the electric acupuncture piece.

11. The therapy device according to claim 2, wherein the machine is provided with an audio signal output port for outputting audio signals generated by the machine.

12. The therapy device according to claim 2, wherein the machine is a machine of the combination of a low-frequency therapy device and a music player.

13. The therapy device according to claim 1, further comprising a conductive patch adhered to the electric acupuncture piece.

14. The therapy device according to claim 2, wherein the machine is a machine of a low-frequency therapy device.

15. The therapy device according to claim 1, wherein the electric acupuncture element further comprises a plug and a lead, the lead is electrically connected to the electric acupuncture piece and the plug.
